(19) United States
(12) Patent Application Publication
(10) Pub. No.: US 2023/0045512 A1
(43) Pub. Date: Feb. 9, 2023

(54) BIOPOWERPLANT: THIRD GENERATION BIOREFINERY WITH IMPROVED CAPACITY TO USE DOMESTIC WASTEWATER, LANDFILL LEACHATE AND SEA SALT WATER AS AN INPUT TO GENERATE GREEN ENERGY, WATER FOR REUSE, BIOFUEL, ORGANIC FERTILIZERS AND CAPTURE ATMOSPHERIC CO2

(71) Applicant: DETECHC LLC, Dover, DE (US)

(72) Inventor: Jaime Eduardo GUTIÉRREZ FONSECA, Bogotá (CO)

(21) Appl. No.: 17/816,291

(22) Filed: Jul. 29, 2022

Related U.S. Application Data
(60) Provisional application No. 63/226,788, filed on Jul. 29, 2021.

(51) Int. Cl. C12M 1/00; C10B 53/02; C10B 57/14; C02F 11/10; C02F 3/32; C02F 3/28; C02F 3/00; C12M 1/107; C12M 1/02; B01J 12/00; C05F 17/90; C05F 7/00; F02C 3/22

(52) U.S. Cl. CPC C12M 43/08; C10B 53/02; C10B 57/14; C02F 11/10; C02F 3/322; C02F 3/2866; C02F 3/005; C12M 21/12; C12M 21/04; C12M 21/02; C12M 43/06; C12M 47/02; C12M 41/18; B01J 12/00; C05F 17/989; C05F 7/00; F02C 3/22; C02F 2103/08

(57) ABSTRACT

The Biopowerplant is a system that integrates the generation of carbon-neutral energy through the cultivation and conversion of microalgal biomass, with sewage sanitation and environmental carbon recovery, with the additional and secondary production of biofertilizer, biofuel, water for reuse. This system integrates a suboptimal anaerobic digestion subsystem focused on the generation of biogas, the processing of the resulting digestate through a microalgal consortium culture subsystem with biofilm induction and smooth decreasing gradient of light radiation, and the transformation of the generated microalgal biomass into syngas through a subsystem of evaporation, torrefaction, pyrolysis, gasification, and combustion in separate chambers. The syngas and methane from the biogas are subsequently used as fuel in an electric power generator capable of operating with mixed gases. The biogas generation process is enriched through the recirculation of the microalgal biomass supernatant, the residual heat from the syngas generation subsystem, and the heat transferred from the combustion gases of the electric generator. The residual sludge from the biogas generation subsystem is recirculated towards a longitudinal biopile subsystem, where it acts as an anaerobic medium compared to the aerobic medium that constitutes the concentrated microalgal biomass, and both streams are mixed to be transformed into the syngas generation subsystem. Input inflows for system operation are mainly sewage, and optionally seawater and/or leachate. The inflows must be bioaugmented with a microalgal consortium dosed automatically by a Compact in situ bioaugmentation system, preferably more than 3 kilometers before the inflow enters the system.

### CROSS-REFERENCE TO RELATED APPLICATIONS

This application claims the benefit of U.S. Provisional Application No. 63/226,788 filed on Jul. 29, 2021, which is incorporated herein by reference in its entirety.

### BACKGROUND OF THE INVENTION

### 1. Field of the Invention

This invention relates to a method and system for the use of domestic wastewater, and/or landfill leachate and/or sea salt water for the generation of carbon neutral electrical energy, the production of water suitable for reuse in agricultural irrigation, industrial applications and drinking water purification, biofuel, and organic fertilizer, simultaneously with environmental carbon recovery. Particularly, this invention relates to a Third generation biorefinery-type system whose operation is the product of the articulated and integrated operation of five subsystems that allow it to use flows from 50 liters per second of domiciliary wastewater, landfill leachate and seawater to cultivate large quantities of biomass from Microalgae-Predominant Microbial Consortium (MPMC) to convert it into fuel gases (syngas and purified biomethane) and from there to carbon neutral electrical energy, recirculating 100% of the CO² generated in the combustion of those gases. This biorefinery system simultaneously converts between 80 and 95% of the delivered flow into water suitable for agricultural irrigation, industrial applications, and even subsequent purification. Additionally, this biorefinery system recovery atmospheric CO² and release O² during the generation of microalgal biomasses.

### 2. Description of the Prior Art

The growth of the human population and the technificiation of society have brought with them a dramatic increase in energy demand and the generation of domestic effluents. Until now, most energy has been produced from non-renewable sources that have increased the concentration of carbon in the atmosphere. In turn, domestic waste is not being treated satisfactorily and a large portion of it is polluting our water sources. To make matters worse, currently installed wastewater treatment systems are mostly energy and chemical intensive, costly to operate and feasible only on large scales.

These issues have stimulated research into environmentally sustainable solutions to generate energy from renewable sources and to increase the effectiveness and coverage of water decontamination systems.

Technologies that exploit the dual capabilities of microalgae to decontaminate water, as well as the energy generation potential of microalgal biomasses and the lipids they produce, were seen as having great potential to meet these needs.

To take advantage of these potentials, the use of microalgae was integrated into biorefineries, which are industrial facilities that seek to obtain several valuable products from one or two biological inputs, using mechanical, thermal, chemical, enzymatic or a mixture of these processes more frequently. The integration of microalgae biotechnology into the biorefinery concept strengthened the capacity of microalgae to be a circular economy or zero waste alternative.

As a circular economy option, biorefinery technologies are receiving a lot of investment in the last five years. Global Investment in New Biorefinery Infrastructure will Total $170 Billion through 2022. And this enthusiasm is not for nothing; biofuels and biochemistry play an important role in the transition to a fossil fuel-free society. This field of technology has one of the highest economic growth potentials in the next 20 years. Among the various technologies, the thermochemical segment will be the fastest growing (17.3% CAGR) during 2015-2020 in the global biorefinery market. The industrial biotechnology segment held the largest market share at $224.8 billion in 2014, and is expected to reach $447.3 billion by 2020, growing at a CAGR of 13.0% during 2015-2020. Most of those plants are dedicated to the valorization of lignocellulosic biomass (LCB) and the generation of biofuels. These biomasses present a challenge for their utilization, requiring costly and energy-demanding pre-treatments, with conversion rates of less than 60%, and must pay costs for chemical inputs, the biomass, and its transportation.

Microalgae biomasses are an important alternative. They are already “liquefied” biomasses that can be produced economically on site and using very few inputs (less than 10% of the cost of lignocellulose ones).

The state of the art in biorefineries based on microalgal biomass is migrating to using sewage as a substrate for microalgae growth. And they focus on the conversion of microalgal biomass into biogas and organic fertilizer. These biorefineries also earn revenue from water, both from charging for sewage decontamination and from selling the decontaminated water for reuse.

The productivity of these biorefineries is still very limited. This is mainly because they do not manage to produce enough biomass per square meter, they still use expensive biomass separation methodologies, and their biomass-to-biogas conversion rates are still inefficient. Additionally, these biorefineries are based on raceway reactors, which are very space demanding, making them unfeasible in regions with high land values. In the case of the figure, a facility with the capacity to treat 1,000 m3 per day (a population of approximately 5,000 people) generates revenues of almost USD 160,000 per year.

This is most likely the main reason why microalgae technologies have so far played a marginal role both in the generation of clean energy and in the care of the planet's water. This is because for microalgae energy applications to be profitable or sustainable, three technological barriers must be overcome: 1. producing more than 25 mg/m2.d of microalgal biomass; 2. recovering (cultivating) more than 90% of this biomass; and 3. converting more than 80% of the recovered biomass directly into energy.

Therefore, an urgent need exists for a novel biorefinery system capable of achieving these three milestones and become an eco-efficient, sustainable, and profitable solution for the generation and commercialization of carbon-negative electric energy, water for reuse, biofuel, organic fertilizer, and atmospheric CO² capture, from the process of using domestic wastewater, landfill leachate and sea salt water.

### BRIEF DESCRIPTION OF DRAWINGS

FIG. 1 is a schematic representation of Biopowerplant biorefinery system according to an exemplary embodiment of the present invention.

FIG. 2 is a schematic representation of Biopowerplant biorefinery system according to another exemplary embodiment of the present invention.

FIG. 3 is a schematic representation of microalgal biomass culture system with biofilm induction and smooth decreasing gradient of light radiation, according to another exemplary embodiment of the present invention.

FIG. 4 is a schematic representation of an evaporation, torrefaction, pyrolysis, gasification, and combustion system, according to another exemplary embodiment of the present invention.

FIG. 5 is a schematic representation of an a concentric microbial cell for hardwater softening, according to another exemplary embodiment of the present invention.

### DETAILED DESCRIPTION OF THE INVENTION

The present invention provides a 3d. class biorefinery for the generation of carbon-neutral energy through the cultivation and conversion of microalgal biomass, with sewage sanitation and environmental carbon recovery, with the additional and secondary production of biofertilizer, biofuel, and water for reuse. An exemplary biorefinery of the invention integrates biomass cultivation with sewage sanitation, the transformation of the generated microalgal biomass into a carbon-neutral electricity and integrates further with environmental carbon recovery from the biomass processing. The processing of the biomass into carbon-neutral electrical energy, biofuels and biofertilizers, through the integration of four processes such as suboptimal anaerobic digestion subsystem, a microalgal aerobic subsystem to treat the digestate, a subsystem for the conversion of the microalgal biomass and sludge from the anaerobic digestion subsystem mixture in syngas, through a sequential process of evaporation, torrefaction, pyrolysis, gasification, and combustion. In addition to electrical energy, the result of the operation of this invention generates an organic phase that is suitable for refining to a biofuel or biofertilizer, and also produces a waste heat stream. The cultivation subsystem includes a biofilm induction structure and smooth decreasing gradient of light radiation to increase the biomass generation to more than 25 mg/m2.d. The biorefinery can optionally comprise a longitudinal biopile cogeneration system, configured to use the concentrated biomass generated in the microalgal aerobic subsystem together with the residual sludge from the suboptimal anaerobic digestion subsystem, as electrical differential generators to produce energy and remove ions from a seawater steam or leachate.

In various embodiments the biorefinery is configured to efficiently recover components from the waste stream from the treatment system, such as residual heat from the syngas generation subsystem, residual heat from the electric power generation subsystem, residual sludge from the suboptimal anaerobic digestion subsystem, the supernatant of the microalgal biomass concentration process. The invention recovery systems can additionally collect waste gases from any other system of the biorefinery, such as the combustion gases from the electricity generation subsystem. In further embodiments molecular hydrogen is a product of the recovery system and is reused in the biorefinery. Molecular hydrogen can also be produced outside of the recovery system, such as through the electrolysis of water.

In additional embodiments, the biorefinery comprises a compact in situ bioaugmentation system of the torrent of inflows with microalgae consortia before the inflow enters the system. In still additional embodiments, the biorefinery comprises a compact in situ bioaugmentation system of the torrent of inflows with microalgae consortia before the inflow enters the system. This bioaugmentation system provides pretreatment to the inflow, preparing it for digestion in the anaerobic digestion subsystem, and increases the proportion of methane in the biogas that will be generated by this subsystem.

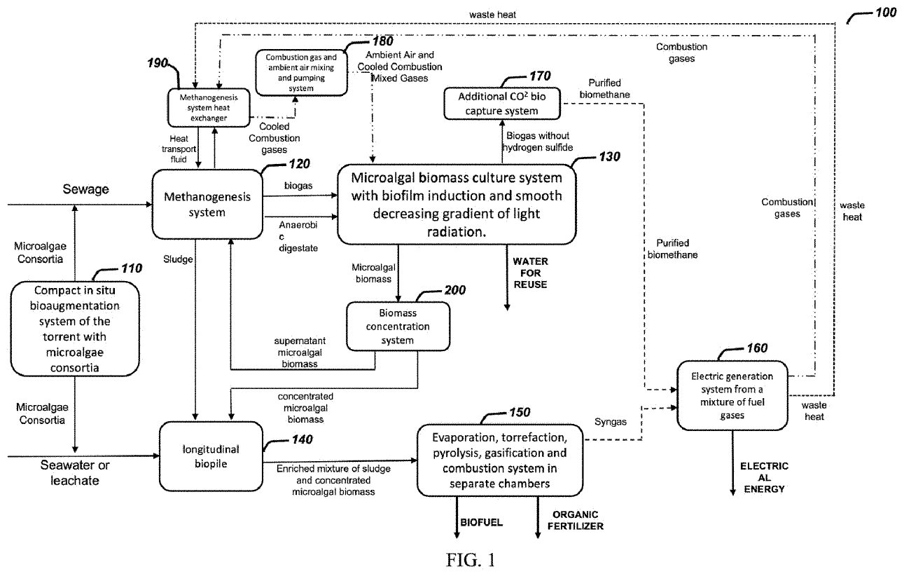

FIG. 1 schematically illustrates core systems of an exemplary Biopowerplant 100 of the present invention. The Biopowerplant 100 mainly comprises a compact in situ bioaugmentation system 110, a methanogenesis system 120, a microalgal biomass culture system with biofilm induction and smooth decreasing gradient of light radiation 130, a longitudinal biopile 140, an evaporation, torrefaction, pyrolysis, gasification, and combustion system 150, and an electric generation system from a mixture of fuel gases 160. The compact in situ bioaugmentation system 110 bioaugments the inflow torrent, either sewage and/or seawater and/or leachate, preferably 3 kilometers before said torrent enters the biopower plant. This torrent is then anaerobically digested by the methanogenesis subsystem 120, from which a liquid digestate and a biogas stream emerge that are injected into the microalgal biomass culture system 130, within which the processes of nutrient consumption and organic load take place. of Microalgae-Predominant Microbial Consortium (MPMC) that grows in it, resulting in the release of water suitable for agricultural and industrial reuse, and the simultaneous production of abundant biomass (more than 25 mg/m2.d), which is directed to the biomass concentration system 200, from where two streams come out, one of biomass clarification, which is recirculated to the methanogenesis system 120, and another of highly concentrated microalgal biomass that is directed to the longitudinal biopile 140, where it feeds the aerobic chamber of said device, and then it is mixed with the residual sludge from the methanogenesis system 120, to form the biomass-sludge mixture that will feed the evaporation, torrefaction, pyrolysis, gasification and combustion system 150, in which this mixture will be converted into syngas, biofuel and biofertilizer. The syngas is directed to the electric generation system from a mixture of fuel gases 160, where it will be used as fuel to generate electricity. For its part, the biogas that was injected from the methanogenesis subsystem 120 to the microalgal biomass culture system 130, has been bubbled through the culture medium so that the Microalgae-Predominant Microbial Consortium (MPMC) removes the hydrogen sulfide, and also the CO² by passing through the Additional CO² bio capture system 170, obtaining more than 90% purified biomethane, which is directed to the electric generation system from a mixture of fuel gases 160, where it will also be used as fuel to generate electricity. The biomass cultivation system 130 also consumes carbon dioxide from the combustion gases generated in the electric generation system from a mixture of fuel gases 160, after being recirculated through the methanogenesis system heat exchanger 190 to take advantage of its residual heat and mix with ambient air in different proportions in the combustion gas and ambient air mixing and pumping system 180. The residual heat generated in the electric generation system from a mixture of fuel gases 160 is also recirculated to the Methanogenesis system heat exchanger 190 to be used in the maintenance of the optimal temperature of the methanogenesis system 120.

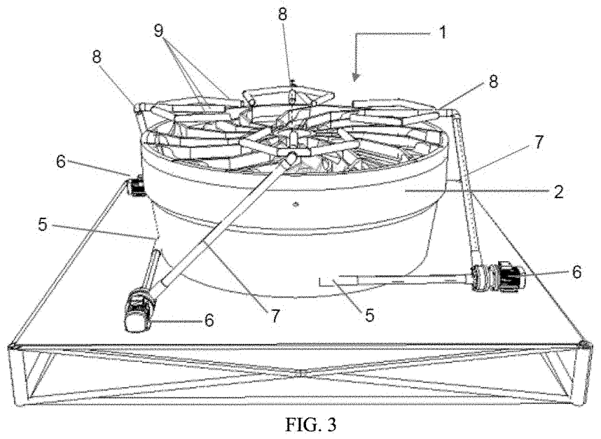

The Microalgal biomass culture system 130 can comprise, for example, a system for culturing Microalgae-Predominant Microbial Consortium (MPMC) (FIG. 3), which comprises an external tank (2) of transparent material with a truncated conical shape, where the diameter of the base is less than the diameter of the upper edge, a support structure for the recirculation mechanism (3), lighting (19) and induced flow, a recirculation mechanism (3), an aeration system (4), an inlet system and effluent outlet, a telemetry and control system and an induced flow system; where the structure of the recirculation mechanism divides the volume of the tank into quadrants and sub-quadrants; The structure of the recirculation mechanism is fixed to the inside of the tank by means of two structures of curved branched arms (11) joined together by membranes (13), inclined at a variable angle with respect to the vertical axis, thanks to the presence of equalizable joints, where forms the film of microorganisms that performs the remediation, and where the lighting of the system is preferably natural.

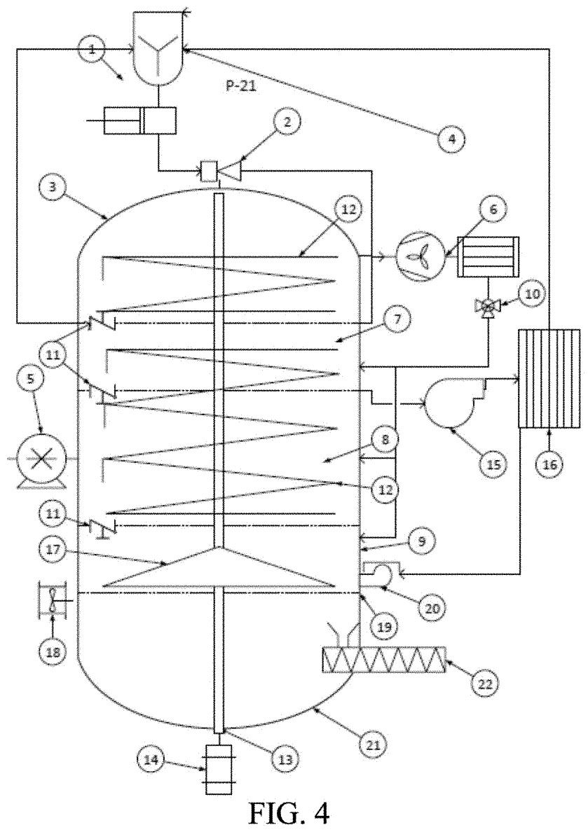

The Evaporation, torrefaction, pyrolysis, gasification and combustion system 150 (FIG. 4) can comprise, for example, a system to transform the residual biomass with moisture up to 80% HBS, or a mixture of said microalgal biomass with residual sludge from the suboptimal anaerobic digestion subsystem, which enters the sludge feed pump 1, which carries the material at a pressure between 7 MPa to 15 MPa and temperature from 50° C. to 200° C., depending on the characteristics of the material. The material passes through a set of adiabatic expansion nozzles 2, which reduces the pressure of the material at the pump outlet, below atmospheric pressure, between 80% and 20% vacuum, producing an instantaneous evaporation or evaporation effect. flash”, which separates the water vapor from the solid fraction in the evaporation chamber 3; the material loses between 10 to 30% of its moisture in each pass through the evaporation chamber where it is heated, so a feedback cycle is required in the pre-mix chamber 4. The vacuum in the rapid evaporation chamber is generated from the torrefaction/pyrolysis chamber by the vacuum pump 5. The steam separated in chamber 3 is fed back to the torrefaction 7/pyrolysis 8 and gasification 9 chambers, through the reheating and ventilation system 6. This steam fed back generates two effects, one of expansion of the bio char activating it and another of increase in the calorific power of the synthesis gas by producing a greater proportion of hydrogen and methane. The steam flow is regulated by means of a three-way metering valve 10. Once the material reaches a humidity of less than 20%, HBS passes to the torrefaction and pyrolysis chamber through the lock-type gate valve 11, which divides the two chambers. the same happens for the passage of the material between the pyrolysis and gasification/combustion chambers, through the gate valves type 11. The material in process, in each chamber is forced to move in an axial direction up-down and from center to center. periphery through the helical agitators 12 that are fixed to the axis of rotation 13, driven by the motor reducer 14. The gases released as volatile matter in the torrefaction/pyrolysis chambers when the material in process exceeds a temperature of 200° C. and up to 450° C., are evacuated by the induced draft fan 15, then pass to the condenser/catalyst 16, where the bio oil precipitates when reaching a temperature below 1 os 60° C. The heat recovered in this system is used in the pre-heating of the sludge, in the pre-mixing chamber 4. The bio char that is generated in the pyrolysis stage in chamber 8, passes to the gasification/combustion chamber 9, Through the gate valve type 11, in chamber 9 the bio char falls on the rotating conical grill 17, where a fraction that does not exceed 30% of the processed volume is gasified and combusts when it comes into contact with the air. primary supplied by the fan 18, to generate the necessary heat in the endothermic reactions of the evaporation, torrefaction, pyrolysis and gasification processes. The heat transfer is carried out between the burned gases and each chamber by means of a jacket 19, insulated towards the outside and radiating towards the inside where the heat flows through the walls and floors to the material in process. The residual heat of the combustion gases at the outlet of the reactor jacket is recovered to preheat the sludge that enters the pre-mix chamber 4. There is also the possibility of using part of the bio-oil for the combustion process, in this In this case, an external burner 20, connected to the heating jacket of the reactor, is additionally placed on the rotating grate. The flow of hot gases in the heating jacket is controlled by butterfly-type flow valves and the retention time of the gases is optimized by using dampers or flow deflector baffles inside the jacket. The bio char and the ashes are cooled in chamber 21 and are removed from the reactor through the auger 22. All the operating variables are controlled by a central electronic system that synchronizes the operations and operational parameters once the performance curves of the reactor for different types of residual biomass. The system is connected to an Internet of Things IoT platform for remote monitoring and operation.

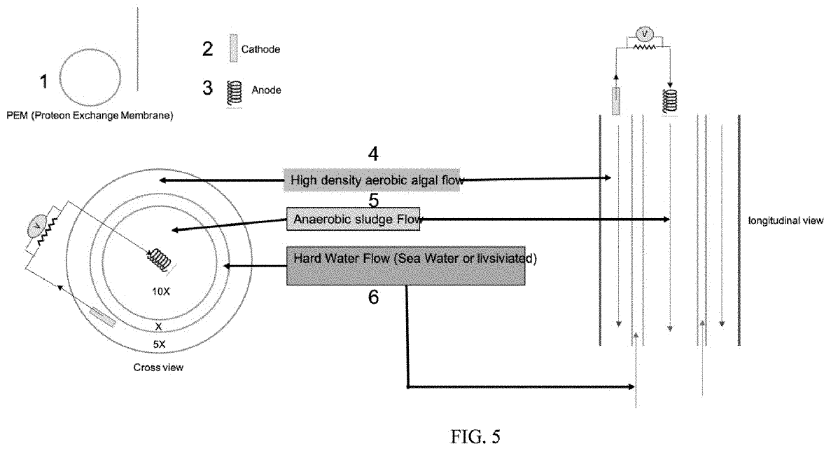

The longitudinal biopile 140 can comprise, for example, a concentric microbial cell for hardwater softening, (FIG. 5), a concentric system of flows contrary to each other, in which two proton exchange membranes (PEM) (1) separate the concentric flows of a torrent of highly concentrated microalgal biomass (4) it comes from the Biomass concentration system 200, an inflow of seawater or leachate, and externally an anaerobic sludge flow (6) which comes from the methanogenesis system 120. The seawater or leachate inflow runs in the opposite direction to the other two. a longitudinal anode (3) is positioned in the center of the anaerobic sludge flow (6), and a longitudinal cathode (2) is located on the external wall of the highly concentrated microalgal biomass torrent compartment (4), between which generates electrical energy that feeds the system.

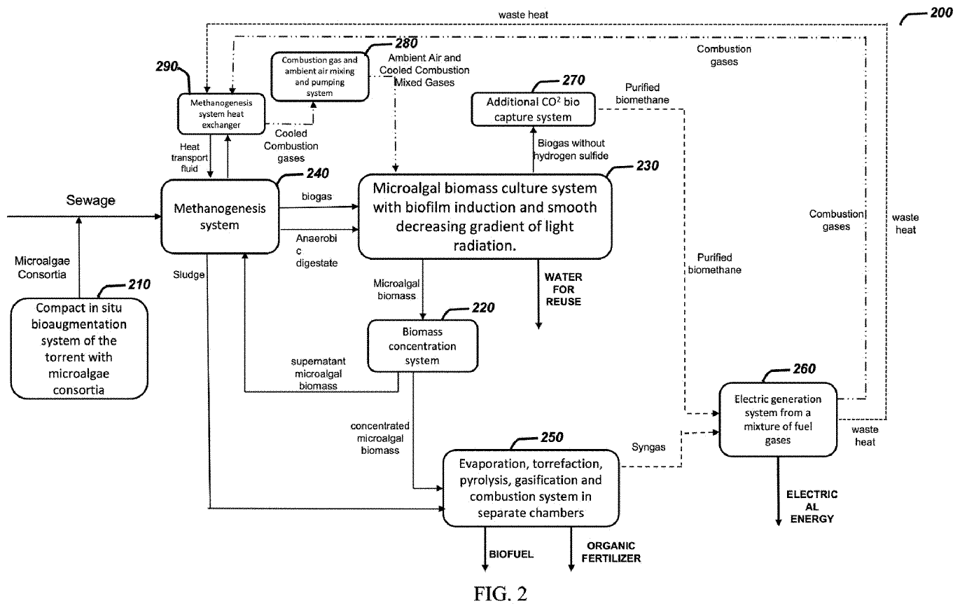

FIG. 2 schematically illustrates another exemplary of Biopowerplant 200 of the present invention. The Biopowerplant 200 mainly comprises a compact in situ bioaugmentation system 210, a methanogenesis system 240, a microalgal biomass culture system with biofilm induction and smooth decreasing gradient of light radiation 230, an evaporation, torrefaction, pyrolysis, gasification, and combustion system 250, and an electric generation system from a mixture of fuel gases 260. The compact in situ bioaugmentation system 210 bioaugments the inflow torrent, either sewage and/or seawater and/or leachate, preferably 3 kilometers before said torrent enters the biopower plant. This torrent is then anaerobically digested by the methanogenesis subsystem 240, from which a liquid digestate and a biogas stream emerge that are injected into the microalgal biomass culture system 230, within which the processes of nutrient consumption and organic load take place. of Microalgae-Predominant Microbial Consortium (MPMC) that grows in it, resulting in the release of water suitable for agricultural and industrial reuse, and the simultaneous production of abundant biomass (more than 25 mg/m2.d), which is directed to the biomass concentration system 220, from where two streams come out, one of biomass clarification, which is recirculated to the methanogenesis system 240, and another of highly concentrated microalgal biomass that is directed to the evaporation, torrefaction, pyrolysis, gasification and combustion system 250, in which this highly concentrated microalgal biomass will be converted into syngas, biofuel and biofertilizer. The syngas is directed to the electric generation system from a mixture of fuel gases 260, where it will be used as fuel to generate electricity. For its part, the biogas that was injected from the methanogenesis subsystem 240 to the microalgal biomass culture system 230, has been bubbled through the culture medium so that the Microalgae-Predominant Microbial Consortium (MPMC) removes the hydrogen sulfide, and also the CO² by passing through the Additional CO² bio capture system 270, obtaining more than 90% purified biomethane, which is directed to the electric generation system from a mixture of fuel gases 260, where it will also be used as fuel to generate electricity. The biomass cultivation system 230 also consumes carbon dioxide from the combustion gases generated in the electric generation system from a mixture of fuel gases 260, after being recirculated through the methanogenesis system heat exchanger 290 to take advantage of its residual heat and mix with ambient air in different proportions in the combustion gas and ambient air mixing and pumping system 280. The residual heat generated in the electric generation system from a mixture of fuel gases 260 is also recirculated to the Methanogenesis system heat exchanger 290 to be used in the maintenance of the optimal temperature of the methanogenesis system 240.

In the description above, the invention is described with reference to specific configurations thereof, but one skilled in the art will recognize that the invention is not limited to just these configurations. Various features and aspects of the invention described above can be used individually or together, and the process can be adjusted to generate different proportions of each by-product. Furthermore, the invention may be implemented in any number of environments and applications beyond those described herein without departing from the broader spirit and scope of the specification. Therefore, the specifications and figures are illustrative elements and never restrictive. It is then stated that the expressions “comprising”, “including” and “having”, used herein, are specifically intended to be read as open terms of art.

What is claimed is:

1. A system, comprising:
a culture system configured to produce biomass by culturing Microalgae-Predominant Microbial Consortium (MPMC) fed with anaerobic digestate from sewage and flue gases;
an electricity generation system from a mixture of syngas obtained from a sequential process of evaporation, torrefaction, pyrolysis, gasification, and combustion of concentrated microalgal biomass and biomethane purified by filtering through the microalgal biomass cultivation system;
an electricity cogeneration system through a concentric microbial cell for hardwater softening; and
a recirculation system configured to recover waste from the system, such as combustion gases, residual heat, anaerobic sludge and microalgal biomass supernatant to recover carbon from the gases, take advantage of the heat in the thermal control of the process and the microalgal biomass supernatant in the increase of methane in biogas from anaerobic digestion.

2. The system of claim 1, wherein the culture system for culturing Microalgae-Predominant Microbial Consortium (MPMC) with a structure of branched arms of biofilm induction and smooth decreasing gradient of light radiation.

3. The system of claim 1, wherein the system comprises the culture system for culturing Microalgae-Predominant Microbial Consortium (MPMC), and a compact in situ inflow bioaugmentation system.

4. The system of claim 1, wherein the recovery system is further configured to recover nutrients from the waste and provide the nutrients to the growing system.

5. The system of claim 1 further comprising a Biomass concentration system.

6. The system of claim 1 further comprising an electricity generation system from a mixture of syngas obtained from a sequential process of evaporation, torrefaction, pyrolysis, gasification, and combustion of concentrated microalgal biomass and purified biomethane.

7. The system of claim 6, wherein the recovery system is further configured to direct gaseous waste from the treatment system to the Methanogenesis system heat exchanger and the Combustion gas and ambient air mixing and pumping system.

8. The system of claim 1, wherein the inflow is anaerobically digested by the methanogenesis.

9. The system of claim 8, further comprising a concentric microbial cell for hardwater softening that uses the concentric flows of a torrent of highly concentrated microalgal biomass and anaerobic sludge flow to decontaminate an inflow of seawater and/or leachate.

10. The system of claim 8, further comprising a hydrogen production system configured to receive at least part of the biogas from the digester and to produce molecular hydrogen from the methane fraction of the biogas.

11. The system of claim 10, wherein the hydrogen production system includes a steam methane reformer.

12. The system of claim 10, wherein the hydrogen production system includes a steam gasifier.

13. The system of claim 1, further comprising a hydrogen production system configured to produce molecular hydrogen by electrolysis of water.

14. The system of claim 1, further comprising:
a carbon dioxide generation system configured to concentrate carbon dioxide from a flue gas stream and air,
a synthesis system configured to receive at least part of the carbon dioxide from the electrical generation system and at least part of the molecular hydrogen from the hydrogen production system.

15. The system of claim 1 further comprising a refining system configured to receive the organic phase from the treatment system and produce a fuel therefrom.

16. The system of claim 1 further configured to provide waste gases from the electric generation system to the hydrogen production system.
